(19) United States
(12) Patent Application Publication
(10) Pub. No.: US 2021/0102947 A1
(43) Pub. Date: Apr. 8, 2021

(54) METHOD TO PERFORM DIAGNOSIS AND PROGNOSIS OF MELANOMA AND KIT

(71) Applicants: Francesco FACCHIANO, Roma (RM) (IT); Antonio FACCHIANO, Roma (RM) (IT); Daniela Agnese D'ARCANGELO, Roma (RM) (IT)

(72) Inventors: Francesco FACCHIANO, Roma (RM) (IT); Antonio FACCHIANO, Roma (RM) (IT); Daniela Agnese D'ARCANGELO, Roma (RM) (IT)

(21) Appl. No.: 17/048,276

(22) PCT Filed: Apr. 16, 2019
(86) PCT No.: PCT/IB2019/053101

§ 371 Date: Oct. 16, 2020

(30) Foreign Application Priority Data
Apr. 17, 2018 (IT) 102018000004609

(51) Int. Cl. G01N 33/574

(52) U.S. Cl. CPC G01N 33/5743; G01N 33/57484

(57) ABSTRACT

The present invention refers to a method to perform a diagnosis of melanoma in a subject, as well as a method to perform a prognosis of a subject suffering from melanoma. Moreover, the present invention refers to a kit which allows to perform a non-invasive diagnosis of melanoma in a subject, and/or to perform a prognosis of a subject suffering from melanoma.

The present invention refers to a method to perform a diagnosis of melanoma in a subject, as well as a method to perform a prognosis of a subject suffering from melanoma. Moreover, the present invention refers to a kit which allows to perform a non-invasive diagnosis of melanoma in a subject, and/or to perform a prognosis of a subject suffering from melanoma.

### STATE OF THE ART

According to data of the American Cancer Society, the skin cancer is the most common kind of tumor. It can originate in each of the three layers that make up the skin, i.e. in the epidermis (outermost layer), in the dermis (intermediate layer) and in the subcutaneous tissue (deep layer).

Among the tumoral lesions of epidermis, melanoma, i.e. the tumoral lesion originating from the melanocytes (melanin-producing cells), is the most dangerous. It can occur in all the body parts where melanocytes are normally present, with a particular preference for the sun-exposed regions.

According to various sources, high rates of melanoma can be found in Oceania, North America, Europe, South Africa and Latin America, i.e. the countries where the exposure to UV light is higher. Moreover, cutaneous melanoma is one of the main tumors occurring at an early age, being classified as the third most frequent tumor in both genders below 50 years old in Italy in 2017. Moreover, AIRTUM (Associazione italiana registri tumori) data identify about 13 cases of melanoma every 100,000 persons, with an estimate being around 3,150 new cases every year among men and 2,850 among women; the melanoma incidence is however steadily increasing, being even doubled in the last 10 years.

In general, diagnosis and prognosis of melanoma are strictly connected: the prognosis, i.e. the prediction of the possible progression of the disease, is better as soon as the tumor is diagnosed. As a matter of fact, the diagnosis earliness increases the probability that the cells of the primary tumor have not formed metastasis, and increases the effectiveness of the treatment suitable to eradicate the primary tumor.

Therefore, the early diagnosis is the most efficient tool in the clinical treatment of melanoma. In fact, when treated in the early stages, the melanomas recover in nearly all the cases and can be often completely cured thanks to surgery. The survival rate at ten years decreases from 95%, when melanoma is diagnosed in the initial stages, to 10%-15% when melanoma is diagnosed in the late metastatic stage.

However, a melanoma diagnosis can be difficult, and in particular an early diagnosis, since a peculiarity of this tumor is the absence of pronounced symptoms in the early stage. Indeed, the only sign of cutaneous melanoma in its early stages is the aspect change of a nevus or the appearance of a new nevus, and such change or appearance can often result difficult to be understood.

To date the presumptive diagnosis of melanoma is carried out with non-invasive procedures (inspection, epiluminescence, confocal microscopy), but such diagnosis has not clinical or legal validity and requires the histological confirmation, which is an invasive, expensive technique, and accomplished in relatively late periods, i.e. when the patient has consulted the specialist physicians. The need for histological diagnosis is connected to the diagnostic reliability, which for the non-invasive techniques (clinical inspection and epiluminescence) does not exceed 80-85% and strongly depends on the operator ability. Non-invasive and quantitative techniques, which can help in the early diagnosis and therefore in relevant reduction of melanoma mortality, are hence required.

Due to the increasing incidence of melanoma, the absence of specific symptoms correlated to its insurgence and the need of performing an early diagnosis, novel sensitive, accurate and non-invasive diagnosis methods are required.

### OBJECTS OF THE INVENTION

Object of the present invention is to provide a method based on the quantitative evaluation of the gene and/or protein expression, which allows to perform a diagnosis of melanoma in a subject, by means of a non-invasive procedure.

Further object of the present invention is to provide a method which allows to perform a prognosis in the case of a subject suffering from melanoma.

Another object of the present invention is to indicate useful molecules to perform the diagnosis of melanoma, and/or to define the prognosis.

### DESCRIPTION OF THE INVENTION

The objects stated above, as well as other objects, are achieved by means of the object of the present invention, i.e. a method to perform a diagnosis of melanoma in a subject, and a method to perform a prognosis of a subject suffering from melanoma, which make use of the quantitative determination of the gene and/or protein expression levels. Such genes and proteins have been identified as expressed in a significantly different way in melanoma samples with respect to healthy skin and/or nevi samples.

Such identification, as it will be extensively described in the experimental section, has been carried out thanks to an analysis of the gene and protein expression on 222 genes selected from database. Such analysis showed that the expression of 42 out of 222 analyzed genes shows a high ability to discriminate the melanomas from the nevi, since they show a significantly different expression. A further analysis revealed that, in particular, 10 genes among the above 42 (in particular, ATG9A, BAG1, CAPN2, CHMP2B, GNAI3, ITGB4, KIAA0226 (also known as RUBCN), MLST8, PEX3, and WIPI1), have never been directly correlated to the melanoma diagnosis.

Therefore, object of the present invention is a method to perform a diagnosis of melanoma in a subject, comprising the determination of the expression levels of at least one gene from at least one sample previously taken and isolated from said subject, characterized in that the at least one gene is selected from the group of genes: ATG9A, BAG1, CAPN2, CHMP2B, GNAI3, ITGB4, KIAA0226, MLST8, PEX3, and WIPI1.

Thanks to a further investigation carried out on the difference in the protein expression in melanomas and healthy skin, some proteins possessing diagnostic value with respect to melanoma have been identified, i.e. the BAG1, PEX3, and WIPI1 proteins.

Therefore, according to the method of the invention to perform a diagnosis, it is possible to determine (in addition to the expression levels of at least one of the 10 genes of the group above, or as an alternative to that) the expression levels of at least one protein encoded by the genes selected from: BAG1, PEX3, and WIPI1.

As it can be observed from the data provided in the experimental section, the genes and the proteins of the above reported lists are significantly reduced or increased in the melanoma with respect to the healthy tissue and/or nevus. For this reason, it is possible to discriminate a sample of melanoma from a sample of healthy skin and/or nevus, on the basis of the expression levels of these genes and/or proteins. Therefore such genes and proteins are clear markers for the diagnosis of melanoma, through their expression levels.

Therefore, in order to perform the diagnosis of melanoma according to the method of the invention, it is possible to determine the expression levels of at least on gene, or more genes, selected from the group of genes: ATG9A, BAG1, CAPN2, CHMP2B, GNAI3, ITGB4, KIAA0226, MLST8, PEX3, and WIPI1. In consequence, it is possible to determine the expression levels of the ATG9A gene, and/or of the BAG1 gene, and/or of the CAPN2 gene, and/or of the CHMP2B gene, and/or of the GNAI3 gene, and/or of the ITGB4 gene, and/or of the KIAA0226 gene, and/or of the MLST8 gene, and/or of the PEX3 gene, and/or of the WIPI1 gene, in order to perform the diagnosis of melanoma according to the method of the invention.

In addition to the determination of the expression levels of the genes selected from the group of genes of the method of the invention to perform a diagnosis, or alternatively to such determination, it is possible to determine the expression levels of at least one protein, or more proteins, selected from the proteins encoded by the following group of genes: BAG1, PEX3 and WIPI1. Therefore, it is possible to determine the expression levels of the protein encoded by the BAG1 gene, and/or of the protein encoded by the PEX3 gene, and/or of the protein encoded by the WIPI1 gene, in order to perform the diagnosis of melanoma according to the method of the invention.

It should be highlighted here the fact that the diagnosis based on the measurement of the expression levels of the above mentioned genes and/or proteins is a quantitative procedure independent from the operator, as set forth in the objects of the invention.

According to the present invention, the expression levels of at least one gene and/or at least one protein can be determined by means of known and standardized in vitro methods. Such in vitro methods for the determination of at least one gene can be, e.g., conventional methods making use of the hybridization of the mRNAs extracted from a sample previously taken and isolated from said subject, or of the correspondent complementary DNA (cDNA), such as for example northern blot, Southern blot, real-time PCR (rtq-PCR), reverse transcriptase PCR (RT-PCR), and microarrays provided with polynucleotide probes. Such in vitro methods for the determination of at least one protein can be, e.g., western blot, microarrays provided with polynucleotide probes, and ELISA.

Advantageously, in the method of the invention the further step of comparing the expression levels obtained by means of the method of the invention with the standard or control expression levels of the same genes and/or the same proteins in healthy controls by means of the standard procedure of ROC analysis is comprised.

The comparison according to the present embodiment allows to establish if the expression of the above indicated gene and/or protein, in the sample taken and isolated from the subject, is compatible with a sample of melanoma or healthy tissue or nevus, and therefore it is possible to diagnose if such subject suffers or not from melanoma. Indeed, the significant difference of the expression of genes and/or proteins in melanomas with respect to the expression of the same genes and/or proteins in the healthy epidermis and/or in the nevi can be a significant indication of melanoma, and therefore such indication can be used to diagnose the tumoral lesion.

In consequence, according to the present invention, it is provided a method to perform a diagnosis of melanoma in a subject, comprising the following steps:
a) determining the expression levels of at least one gene, and/or of at least one protein encoded by at least one gene, from one or more samples taken and isolated from said subject; and
b) comparing said expression levels determined in step a), with the standard or control expression levels of the same genes and/or the same proteins, measured in healthy subjects
   characterized in that said at least one gene is selected from the group of genes: ATG9A, BAG1, CAPN2, CHMP2B, GNAI3, ITGB4, KIAA0226, MLST8, PEX3, and WIPI1, and/or said at least one protein is a protein encoded by the genes selected from the group of genes: BAG1, PEX3, and WIPI1.

According to the present invention, with “the standard or control expression levels of the same genes and/or the same proteins” it is meant the expression levels of said genes and/or proteins in healthy controls of reference. Such standard or control expression levels can be, e.g., a threshold value previously determined starting from the mean of the expression levels (obtained from several healthy and/or nevi samples) of a given gene. Such standard or control expression levels can be determined, e.g., starting from isolated samples of healthy tissue (i.e., tissue not affected by melanoma, preferably of a healthy subject, i.e. not suffering from melanoma) or of nevus, and such isolated samples can be, preferably, of healthy epidermis or nevus. Such standard or control expression levels can be alternatively extracted from databases of various kinds.

Moreover it has been surprisingly found, thanks to a validation test described in the experimental section, that 4 genes (among the group of the 10 genes described above) are differently expressed in a particular way in melanomas with respect to healthy skin. These 4 genes are BAG1, CHMP2B, PEX3 and WIPI1.

Therefore, according to the present invention, the at least one gene of which the expression levels are determined is preferably selected from the group of genes consisting of: BAG1, CHMP2B, PEX3, and WIPI1. The determination of the expression of the BAG1 gene, and/or of the CHMP2B gene, and/or of the PEX3 gene, and/or of the WIPI1 gene is particularly advantageous and significant for the in vitro diagnosis of melanoma in a subject suspecting of suffering from melanoma.

Moreover, by means of the analyses described in the experimental section, the prognostic value of the 4 genes identified following the above mentioned validation test has been evaluated. From such analyses it turned out that the expression levels of 2 genes out of 4 (i.e., BAG1 and WIPI1) may be directly correlated to the disease trend, in particular with respect to the survival free from progression of the disease. As a consequence, BAG1 and WIPI1 can be used as markers in in vitro prognosis methods.

Therefore, it is further an object of the present invention a method to perform a prognosis for a subject suffering from melanoma, comprising the determination of the expression levels of at least one gene and/or at least one protein from one or more samples taken and isolated from the subject, characterized in that the at least on gene is selected from the BAG1 and WIPI1 genes, and/or the at least one protein is a protein encoded by the genes selected from BAG1 and WIPI1.

In order to perform a prognosis of a patient suffering from melanoma, it is therefore possible to determine the expression levels of the BAG1 gene, and/or of the WIPI1 gene, and/or of the protein encoded by the BAG1 gene, and/or of the protein encoded by the WIPI1 gene.

The BAG1 and WIPI1 genes, as well as the proteins encoded by them, being significant in the melanoma prognosis, are also useful in order to determine the type of therapeutic treatment to which the patient suffering from melanoma will have to undergo. Indeed, on the basis of the prognosis formulated also thanks to the in vitro determination of at least one gene selected from the BAG1 and WIPI1 genes and/or of at least one protein encoded by the genes selected from BAG1 and WIPI1 (according to the method of the invention to perform a prognosis), it is possible to identify the most appropriate therapy for such patient.

Advantageously, in the method of the invention to perform a prognosis it is further comprised the step of comparing the expression levels determined by means of the method of the invention to perform a prognosis with the standard or control expression levels of the same genes and/or the same proteins.

Such comparison will allow to make the best prognosis of the trend of the melanoma in said subject suffering from melanoma, as well as it will allow to select the most appropriate therapeutic treatment for said subject.

Therefore, it is provided a method to perform a prognosis for a subject suffering from melanoma, comprising the following steps:
a′) determining the expression levels of at least one gene, and/or of at least one protein encoded by a gene, from at least one sample taken and isolated from said subject; and
b′) comparing said expression levels determined in step a′), with the standard or control expression levels of the same genes and/or the same proteins
   characterized in that said at least one gene is selected from the BAG1 and WIPI1 genes, and/or said at least one protein is a protein encoded by the genes selected from BAG1 and WIPI1.

Moreover it has been surprisingly found that, among all the analyzed genes, only the expression levels of WIPI1 are significantly increased in the melanoma samples with respect to the samples of healthy skin, both in terms of gene transcription and of protein expression (and this can be observed thanks to the data reported in the experimental section). To such differential expression of WIPI1 it is possible to attribute both a diagnostic value and a prognostic value. Therefore, in the method of the invention to perform a diagnosis and in the method of the invention to perform a prognosis, the at least one gene of which the expression levels are determined and/or the protein of which the expression levels are determined is preferably WIPI1.

Since the significant differential expression of the genes and/or proteins above, having both a diagnostic and a prognostic value, has been found in isolated samples of melanoma with respect to isolated samples of epidermis and also of nevi, the at least one isolated sample in the methods of the invention is preferably at least one sample of isolated epidermis, more preferably it is at least one sample of isolated nevus. Therefore, when said isolated sample is a nevus sample, it will be possible to effectively diagnose a melanoma also starting from its early onset stages, i.e. when it is still asymptomatic and the only relevant sign is the change of a nevus or the onset of a new one.

Indeed, the measurement of WIPI1 is representative of the presence of melanoma when the analysis is performed on the blood, by means of a routine blood test (and therefore also where there has not been a specific indication by the dermatologist to the removal of a suspect nevus).

A further object of the present invention is a diagnostic kit comprising one or more agents suitable to measure the expression levels of at least one gene and/or at least one protein, characterized in that the at least one gene is selected from the group of genes: ATG9A, BAG1, CAPN2, CHMP2B, GNAI3, ITGB4, KIAA0226, MLST8, PEX3, and WIPI1, and/or the at least one protein is a protein encoded by the genes selected from the group of genes: BAG1, PEX3, and WIPI1.

The kit of the invention is useful to provide a diagnosis in subjects suspecting of suffering from melanoma and/or a prognosis in subjects suffering from melanoma, since it comprises the agents necessary for the measurement, i.e. the determination, of the expression levels of at least one gene and/or at least one protein of the above mentioned groups. Such genes and proteins, as already extensively mentioned, have been identified as differently expressed in a significant way in melanomas with respect to the correspondent expression in healthy tissues and/or in nevi. Therefore, the kit of the invention allows to implement the methods (both of diagnosis and prognosis) object of the present invention.

According to the present invention, the one or more agent are markers which are conventionally used for the determination of the expression levels of genes and/or proteins encoded by them. By way of example, such agents can be markers of polynucleotide nature and/or peptide nature, and can be, still by way of example, protein or DNA or RNA probes, antibodies, etc.

These one or more agents are then agents which allow to determine the expression levels of genes and/or proteins based on known procedures for the determination of the expression levels of genes and/or proteins, such as for example Southern blot, northern blot, rtq-PCR, RT-PCR, DNA microarray, western blot and protein microarrays. The role of such one or more agents is therefore to allow the expression levels of at least one gene selected from the group of genes to be determined: ATG9A, BAG1, CAPN2, CHMP2B, GNAI3, ITGB4, KIAA0226, MLST8, PEX3, and WIPI1, and/or of at least one protein encoded by the genes selected from the group of genes: BAG1, PEX3, and WIPI1.

Therefore, the kit of the invention can comprise one or more agents which allow the measurement, i.e. the determination, of the expression levels of at least one gene, or more genes, selected from the group of genes: ATG9A, BAG1, CAPN2, CHMP2B, GNAI3, ITGB4, KIAA0226, MLST8, PEX3, and WIPI1. Moreover, or alternatively, the kit of the invention can comprise one or more agents which allow the measurement, i.e. the determination, of the expression levels of at least one protein, or more proteins, encoded by the group of genes: BAG1, PEX3, and WIPI1.

According to the kit of the present invention, the at least one gene is advantageously selected from the group of genes: BAG1, CHMP2B, PEX3, and WIPI1. Such kit is particularly useful to perform a diagnosis of a subject suspected of suffering from melanoma since, as already described and as it can be observed in the experimental section, such 3 genes are expressed in a significantly different way in the melanoma with respect to the correspondent expression in the healthy subject, also in view of validation tests. Therefore, a kit which allows the measurement, i.e. the determination of the expression levels of the BAG1 gene, and/or of the CHMP2B gene, and/or of the PEX3 gene, and/or of the WIPI1 gene, is particularly advantageous to perform an in vitro diagnosis of melanoma, starting from at least one sample taken and isolated from said subject.

Alternatively, according to the kit of the present invention, the at least one gene is advantageously selected from the BAG1 and WIPI1 genes, and/or the at least one protein is selected from the proteins encoded by the BAG1 and WIPI1 genes. Since BAG1 and WIPI1 have shown a prognostic value (as already described and observable in the experimental section), the kit allowing the measurement of the expression levels of such 2 genes, as well as of the proteins encoded by them, will be useful to perform a prognosis for a subject suffering from melanoma.

Still according to the invention, the kit advantageously comprises reagents apt to measure the expression levels of at least one gene and/or at least one protein.

The reagents are reagents which are conventionally used in the procedures known for the determination of the expression levels of genes, e.g. in the procedures stated above, and their function is to make possible the implementation of the procedure to measure, i.e. to determine, the expression levels of at least one gene selected from the group of genes: ATG9A, BAG1, CAPN2, CHMP2B, GNAI3, ITGB4, KIAA0226, MLST8, PEX3, and WIPI1, and/or of at least one protein encoded by the genes selected from the group of genes: BAG1, PEX3, and WIPI1.

### DESCRIPTION OF THE FIGURES

FIG. 1 shows some plots where the area under the curve (AUC) of each gene differentially expressed in the melanoma with respect to controls, is reported as sensitivity percentage (Sensitivity %) vs specificity percentage (100-specificity %); the AUC calculated for each gene is reported with a p value <0.0001. The data refer to the expression measurements reported in the GEO public database (https://www.ncbi.nlm.nih.gov/gds)

FIG. 2 reports the gene expression according to the IST Online public database (http://ist.medisapiens.com/). In particular, this figure reports the differential expression levels in melanoma vs healthy skin of 208 melanoma biopsies and of 147 healthy skin biopsies. The dotted lines depict the discriminant level of 90% of the melanoma samples from the remaining 10% of the melanoma samples. The expression of PEX3, BAG1 and CHMP2B in the melanomas is lower with respect to the expression measured in the normal skin in at least 90% of the melanoma cases, while the expression of WIPI1 in the melanomas is higher than the expression measured in the healthy controls in at least 90% of the melanoma cases.

FIG. 3 is the expression of the proteins encoded by the BAG1, PEX3 and WIPI1 genes in the Human Protein Atlas public database (https://www.proteinatlas.org).

The histological sections reported in the database have been obtained and converted in grayscale; the pixels have been then quantified as a function of their gray intensity according to an intensity scale ranging from the lightest gray (value of 255, corresponding to the lowest expression) to the darkest gray (0 value, corresponding to the greatest expression). The Figure shows that the distribution medians are shifted to the right in the BAG1, PEX3 and WIPI1 cases, suggesting for these proteins an expression in the melanoma greater than the controls.

### Experimental Section

In the present experimental section will be reported, in the order as they have been performed, the tests and the analyses which allowed to identify the genes and the proteins differently expressed in a significant way in the melanomas with respect to the corresponding expression in the tissues of healthy subjects.

### Example 1

### Comparative Analysis of the Expression of Genes Correlated to the Autophagy (ARGs) in Melanoma Vs Normal Skin Samples

To identify potential genes and proteins involved in the development of melanoma and in its progression, 222 genes correlated to autophagy (ARGs) have been selected from the Human Autophagy Database (HADb) (http://www.autophagy.lu) to be studied.

The difference between the expression level of melanoma samples and of healthy epidermis samples of these 222 ARGs has been evaluated starting from 3 datasets (Talantov, Riker e Haqq) on the Oncomine database (www.oncomine.org) on the basis of 110 samples of melanoma and 15 samples of normal skin (125 total samples). Then the number of times where the mean value of the expression level of each of the 222 genes in the 110 melanoma samples is higher or lower than the mean value of the expression level of the same gene in the 15 samples of normal skin (fold change difference) has been calculated. Such calculation has been performed as follows:

FC difference=2^{d}

wherein d=X₂−X₁; X₂ is the mean of the values relative to the expression level of the gene of interest in the samples of condition 1 (melanoma); and X₁ is the mean of the values relative to the expression level of the gene of interest in the samples of condition 2 (normal skin). In the present case, X₂ is the mean of the values relative to the expression level of the gene of interest in the 110 samples of melanoma (condition 1) and X₁ is the mean of the values relative to expression level of the gene of interest in the 15 control samples (condition 2).

The fold change difference highlighted that, in melanoma, 70 ARGs out of 222 show significant up-regulation or down-regulation, i.e. greater than +1.5 or lower than −1.5, with respect to the healthy skin. In Table 1 (depicted below) there is the list of the 222 analyzed ARGs and the fold change difference values; the 70 genes showing significant up-regulation or down-regulation are indicated by an x.

TABLE 1

| | Name of | Symbol of | Fold change |
|---|---|---|---|
| # | the gene | the gene | difference |
| 1 | autophagy/beclin-1 regulator 1 | AMBRA1 | 1.41 |
| 2 | apolipoprotein L, 1 | APOL1 | −0.11 |
| 3 | aryl hydrocarbon receptor nuclear | ARNT | 0.70 |
| | translocator | | |
| 4 | arylsulfatase A | ARSA | 0.67 |
| 5 | arylsulfatase B | ARSB | 0.11 |
| 6 | activating transcription factor 4 | ATF4 | 0.50 |
| 7 | activating transcription factor 6 | ATF6 | 0.25 |
| 8 | ATG10 autophagy related 10 | ATG10 | −0.11 |
| | homolog (S. cerevisiae) | | |
| 9 | ATG12 autophagy related 12 | ATG12 | 1.11 |
| | homolog (S. cerevisiae) | | |
| 10 | ATG16 autophagy related | ATG16L1 | 0.91 |
| | 16-like 1 (S. cerevisiae) | | |
| 11x | ATG16 autophagy related | ATG16L2 | −2.21 |
| | 16-like 2 (S. cerevisiae) | | |
| 12 | ATG2 autophagy related 2 | ATG2A | −1.19 |
| | homolog A (S. cerevisiae) | | |
| 13 | ATG2 autophagy related 2 | ATG2B | −0.27 |
| | homolog B (S. cerevisiae) | | |
| 14 | ATG3 autophagy related 3 | ATG3 | 0.58 |
| | homolog (S. cerevisiae) | | |
| 15 | ATG4 autophagy related 4 | ATG4A | |
| | homolog A (S. cerevisiae) | | |
| 16 | ATG4 autophagy related 4 | ATG4B | 1.44 |
| | homolog B (S. cerevisiae) | | |
| 17 | ATG4 autophagy related 4 | ATG4C | −0.08 |
| | homolog C (S. cerevisiae) | | |
| 18 | ATG4 autophagy related 4 | ATG4D | 1.03 |
| | homolog D (S. cerevisiae) | | |
| 19 | ATG5 autophagy related 5 | ATG5 | 0.95 |
| | homolog (S. cerevisiae) | | |
| 20 | ATG7 autophagy related 7 | ATG7 | 0.60 |
| | homolog (S. cerevisiae) | | |
| 21 | ATG9 autophagy related 9 | ATG9A | 0.61 |
| | homolog A (S. cerevisiae) | | |
| 22 | ATG9 autophagy related 9 | ATG9B | −0.63 |
| | homolog B (S. cerevisiae) | | |
| 23 | 5-aminoimidazole-4- | ATIC | 1.08 |
| | carboxamide ribonucleotide | | |
| | formyltransferase/IMP | | |
| | cyclohydrolase | | |
| 24 | BCL2-associated athanogene | BAG1 | −1.43 |
| 25 | BCL2-associated athanogene 3 | BAG3 | 0.04 |
| 26 | BCL2-antagonist/killer 1 | BAK1 | 0.89 |
| 27x | BCL2-associated X protein | BAX | 8.16 |
| 28 | B-cell CLL/lymphoma 2 | BCL2 | 0.71 |
| 29x | BCL2-like 1 | BCL2L1 | 1.86 |
| 30x | beclin 1, autophagy related | BECN1 | 1.58 |
| 31 | BH3 interacting domain death | BID | 0.80 |
| | agonist | | |
| 32x | baculoviral IAP repeat-contain- | BIRC5 | 3.57 |
| | ing 5 | | |
| 33 | baculoviral IAP repeat-contain- | BIRC6 | −0.39 |
| | ing 6 | | |
| 34 | BCL2/adenovirus E1B 19 kDa | BNIP1 | 1.24 |
| | interacting protein 1 | | |
| 35 | BCL2/adenovirus E1B 19 kDa | BNIP3 | −0.11 |
| | interacting protein 3 | | |
| 36 | BCL2/adenovirus E1B 19 kDa | BNIP3L | 0.00 |
| | interacting protein 3-like | | |
| 37 | chromosome 12 open reading | C12orf44 | 0.67 |
| | frame 44 | | |
| 38 | chromosome 17 open reading | C17orf88 | 1.01 |
| | frame 88 | | |
| 39 | calcium binding and | CALCOCO2 | −0.35 |
| | coiled-coil domain 2 | | |
| 40 | calcium/calmodulin- | CAMKK2 | 1.49 |
| | dependent protein kinase | | |
| | kinase 2, beta | | |
| 41x | calnexin | CANX | 1.97 |
| 42 | calpain 1, (mu/I) large | CAPN1 | −0.82 |
| | subunit | | |
| 43 | calpain 10 | CAPN10 | 0.49 |
| 44x | calpain 2, (m/II) large | CAPN2 | 1.50 |
| | subunit | | |
| 45x | calpain, small subunit 1 | CAPNS1 | 3.29 |
| 46 | caspase 1, apoptosis- | CASP1 | 0.84 |
| | related cysteine peptidase | | |
| | (interleukin 1, beta, | | |
| | convertase) | | |
| 47 | caspase 3, apoptosis-related | CASP3 | 0.76 |
| | cysteine peptidase | | |
| 48 | caspase 4, apoptosis-related | CASP4 | 0.58 |
| | cysteine peptidase | | |
| 49 | caspase 8, apoptosis-related | CASP8 | 0.47 |
| | cysteine peptidase | | |
| 50 | chemokine (C-C motif) | CCL2 | −0.57 |
| | ligand 2 | | |
| 51 | chemokine (C-C motif) | CCR2 | |
| | receptor 2 | | |
| 52 | CD46 molecule, complement | CD46 | 1.39 |
| | regulatory protein | | |
| 53 | cyclin-dependent kinase | CDKN1A | 1.15 |
| | inhibitor 1A (p21, Cip1) | | |
| 54 | cyclin-dependent kinase | CDKN1B | −0.64 |
| | inhibitor 1B (p27, Kip1) | | |
| 55x | cyclin-dependent kinase | CDKN2A | 2.36 |
| | inhibitor 2A (melanoma, | | |
| | p16, inhibits CDK4) | | |
| 56 | CASP8 and FADD-like apoptosis | CFLAR | 1.17 |
| | regulator | | |
| 57 | chromatin modifying protein 2B | CHMP2B | −0.17 |
| 58 | chromatin modifying protein 4B | CHMP4B | −1.12 |
| 59x | ceroid-lipofuscinosis, | CLN3 | 1.51 |
| | neuronal 3 | | |
| 60x | cathepsin B | CTSB | 7.17 |
| 61 | cathepsin D | CTSD | 0.86 |
| 62 | cathepsin L1 | CTSL1 | 0.89 |
| 63x | chemokine (C-X3-C motif) | CX3CL1 | −1.96 |
| | ligand 1 | | |
| 64x | chemokine (C-X-C motif) | CXCR4 | 2.79 |
| | receptor 4 | | |
| 65 | death-associated protein | DAPK1 | 1.15 |
| | kinase 1 | | |
| 66x | death-associated protein | DAPK2 | −1.56 |
| | kinase 2 | | |
| 67 | DNA-damage-inducible | DDIT3 | 0.23 |
| | transcript 3 | | |
| 68x | DIRAS family, GTP-binding | DIRAS3 | −1.84 |
| | RAS-like 3 | | |
| 69x | deleted in liver cancer 1 | DLC1 | 1.68 |
| 70 | DnaJ (Hsp40) homolog, | DNAJB1 | 0.82 |
| | subfamily B, member 1 | | |
| 71 | DnaJ (Hsp40) homolog, | DNAJB9 | 1.47 |
| | subfamily B, member 9 | | |
| 72 | DNA-damage regulated | DRAM1 | 0.11 |
| | autophagy modulator 1 | | |
| 73 | ER degradation enhancer, | EDEM1 | 0.42 |
| | mannosidase alpha-like 1 | | |
| 74 | eukaryotic translation | EEF2 | −0.29 |
| | elongation factor 2 | | |
| 75x | eukaryotic elongation | EEF2K | −3.22 |
| | factor-2 kinase | | |
| 76 | epidermal growth factor | EGFR | −1.47 |
| | receptor | | |
| 77 | eukaryotic translation | EIF2AK2 | 1.44 |
| | initiation factor 2-alpha | | |
| | kinase 2 | | |
| 78 | eukaryotic translation | EIF2AK3 | 1.10 |
| | initiation factor 2-alpha | | |
| | kinase 3 | | |
| 79 | eukaryotic translation | EIF2S1 | 0.19 |
| | initiation factor 2, | | |
| | subunit 1 alpha, 35 kDa | | |
| 80x | eukaryotic translation | EIF4EBP1 | 2.78 |
| | initiation factor 4E | | |
| | binding protein 1 | | |
| 81x | eukaryotic translation | EIF4G1 | 3.67 |
| | initiation factor 4 | | |
| | gamma, 1 | | |
| 82 | v-erb-b2 erythroblastic | ERBB2 | −0.16 |
| | leukemia viral oncogene | | |
| | homolog 2, neuro/glioblas- | | |
| | toma derived oncogene | | |
| | homolog (avian) | | |
| 83 | endoplasmic reticulum to | ERN1 | 1.21 |
| | nucleus signaling 1 | | |
| 84x | ERO1-like (S. cerevisiae) | ERO1L | 1.74 |
| 85 | Fas (TNFRSF6)-associated | FADD | 1.47 |
| | via death domain | | |
| 86 | family with sequence | FAM48A | 0.35 |
| | similarity 48, member A | | |
| 87 | Fas (TNF receptor super- | FAS | 1.35 |
| | family, member 6) | | |
| 88x | FK506 binding protein 1A, | FKBP1A | 1.82 |
| | 12 kDa | | |
| 89x | FK506 binding protein 1B, | FKBP1B | 1.76 |
| | 12.6 kDa | | |
| 90x | FBJ murine osteosarcoma | FOS | −5.84 |
| | viral oncogene homolog | | |
| 91 | forkhead box O1 | FOXO1 | −0.52 |
| 92 | forkhead box O3 | FOXO3 | −0.67 |
| 93x | glucosidase, alpha; acid | GAA | 2.02 |
| 94 | GABA(A) receptor-associated | GABARAP | 0.91 |
| | protein | | |
| 95 | GABA(A) receptor-associated | GABARAPL1 | 0.57 |
| | protein like 1 | | |
| 96 | GABA(A) receptor-associated | GABARAPL2 | 0.54 |
| | protein-like 2 | | |
| 97x | glyceraldehyde-3-phosphate | GAPDH | 3.17 |
| | dehydrogenase | | |
| 98 | guanine nucleotide binding | GNAI3 | −0.26 |
| | protein (G protein), alpha | | |
| | inhibiting activity | | |
| | polypeptide 3 | | |
| 99 | guanine nucleotide binding | GNB2L1 | 1.48 |
| | protein (G protein), beta | | |
| | polypeptide 2-like 1 | | |
| 100 | golgi-associated PDZ and | GOPC | 1.41 |
| | coiled-coil motif containing | | |
| 101x | glutamate receptor, ionotropic, | GRID1 | 1.93 |
| | delta 1 | | |
| 102 | glutamate receptor, ionotropic, | GRID2 | 0.11 |
| | delta 2 | | |
| 103 | histone deacetylase 1 | HDAC1 | 1.43 |
| 104 | histone deacetylase 6 | HDAC6 | 1.44 |
| 105 | hepatocyte growth factor- | HGS | 1.34 |
| | regulated tyrosine kinase | | |
| | substrate | | |
| 106 | hypoxia inducible factor 1, | HIF1A | 0.15 |
| | alpha subunit (basic helix- | | |
| | loop-helix transcription | | |
| | factor) | | |
| 107x | heat shock protein 90 kDa alpha | HSP90AB1 | 2.64 |
| | (cytosolic), class B member 1 | | |
| 108x | heat shock 70 kDa protein 5 | HSPA5 | 2.08 |
| | (glucose-regulated protein, | | |
| | 78 kDa) | | |
| 109x | heat shock 70 kDa protein 8 | HSPA8 | 4.53 |
| 110x | heat shock 22 kDa protein 8 | HSPB8 | −3.99 |
| 111 | interferon, gamma | IFNG | not found |
| 112x | inhibitor of kappa light | IKBKB | 2.00 |
| | polypeptide gene enhancer | | |
| | in B-cells, kinase beta | | |
| 113 | inhibitor of kappa light | IKBKE | 1.24 |
| | polypeptide gene enhancer | | |
| | in B-cells, kinase epsilon | | |
| 114x | interleukin 24 | IL24 | 2.08 |
| 115 | immunity-related GTPase | IRGM | −1.41 |
| | family, M | | |
| 116x | integrin, alpha 3 (antigen | ITGA3 | 5.18 |
| | CD49C, alpha 3 subunit of | | |
| | VLA-3 receptor) | | |
| 117x | integrin, alpha 6 | ITGA6 | 4.31 |
| 118x | integrin, beta 1 (fibronectin | ITGB1 | 2.63 |
| | receptor, beta polypeptide, | | |
| | antigen CD29 includes MDF2, | | |
| | MSK12) | | |
| 119x | integrin, beta 4 | ITGB4 | 1.84 |
| 120 | inositol 1,4,5-triphosphate | ITPR1 | 0.06 |
| | receptor, type 1 | | |
| 121 | KIAA0226 | KIAA0226 | 0.07 |
| 122 | KIAA0652 | KIAA0652 | 1.30 |
| 123 | KIAA0831 | KIAA0831 | −1.13 |
| 124 | kinesin family member 5B | KIF5B | −0.51 |
| 125x | kelch-like 24 (Drosophila) | KLHL24 | 1.95 |
| 126x | lysosomal-associated membrane | LAMP1 | 2.28 |
| | protein 1 | | |
| 127 | lysosomal-associated membrane | LAMP2 | 0.63 |
| | protein 2 | | |
| 128 | microtubule-associated protein 1 | MAP1LC3A | −1.10 |
| | light chain 3 alpha | | |
| 128x | microtubule-associated protein 1 | MAP1LC3B | 1.94 |
| | light chain 3 beta | | |
| 130 | microtubule-associated protein 1 | MAP1LC3C | −1.22 |
| | light chain 3 gamma | | |
| 131 | mitogen-activated protein kinase | MAP2K7 | 0.82 |
| | kinase 7 | | |
| 132x | mitogen-activated protein kinase 1 | MAPK1 | 1.52 |
| 133 | mitogen-activated protein kinase 3 | MAPK3 | −0.08 |
| 134 | mitogen-activated protein kinase 8 | MAPK8 | 0.88 |
| 135 | mitogen-activated protein kinase 8 | MAPK8IP1 | 0.96 |
| | interacting protein 1 | | |
| 136 | mitogen-activated protein kinase 9 | MAPK9 | 1.41 |
| 137x | membrane-bound transcription | MBTPS2 | 1.98 |
| | factor peptidase, site 2 | | |
| 138x | MTOR associated protein, LST8 | MLST8 | 5.05 |
| | homolog (S. cerevisiae) | | |
| 139 | myotubularin related protein 14 | MTMR14 | 0.12 |
| 140 | mechanistic target of rapamycin | MTOR | −0.03 |
| | (serine/threonine kinase) | | |
| 141x | v-myc myelocytomatosis viral | MYC | 2.60 |
| | oncogene homolog (avian) | | |
| 142x | nuclear assembly factor 1 homolog | NAF1 | −1.56 |
| | (S. cerevisiae) | | |
| 143 | nicotinamide phosphoribosyl- | NAMPT | 1.11 |
| | transferase | | |
| 144 | neighbor of BRCA1 gene 1 | NBR1 | 1.23 |
| 145 | NCK-associated protein 1 | NCKAP1 | −0.38 |
| 146 | nuclear factor (erythroid-derived | NFE2L2 | 0.55 |
| | 2)-like 2 | | |
| 147 | nuclear factor of k light polypep- | NFKB1 | −0.54 |
| | tide gene enhancer in B-cells 1 | | |
| 148 | NK2 transcription factor related, | NKX2-3 | 0.26 |
| | locus 3 (Drosophila) | | |
| 149x | NLR family, CARD domain | NLRC4 | 1.84 |
| | containing 4 | | |
| 150x | Niemann-Pick disease, type C1 | NPC1 | 1.93 |
| 151 | neuregulin 1 | NRG1 | 0.60 |
| 152 | neuregulin 2 | NRG2 | 0.31 |
| 153 | neuregulin 3 | NRG3 | −1.14 |
| 154x | prolyl 4-hydroxylase, beta | P4HB | 3.67 |
| | polypeptide | | |
| 155 | Parkinson disease (autosomal | PARK2 | 1.28 |
| | recessive, juvenile) 2, parkin | | |
| 156x | poly (ADP-ribose) polymerase 1 | PARP1 | 2.61 |
| 157x | phosphoprotein enriched in astro- | PEA15 | 2.26 |
| | cytes 15 | | |
| 158 | proline, glutamate and leucine | PELP1 | −1.18 |
| | rich protein 1 | | |
| 159 | peroxisomal biogenesis factor 14 | PEX14 | 1.36 |
| 160 | peroxisomal biogenesis factor 3 | PEX3 | 0.20 |
| 161 | phosphoinositide-3-kinase, class 3 | PIK3C3 | 0.63 |
| 162 | phosphoinositide-3-kinase, | PIK3R4 | 0.82 |
| | regulatory subunit 4 | | |
| 163 | PTEN induced putative kinase 1 | PINK1 | −0.77 |
| 164 | protein phosphatase 1, regulatory | PPP1R15A | 1.09 |
| | (inhibitor) subunit 15A | | |
| 165 | protein kinase, AMP-activated, | PRKAB1 | 0.42 |
| | beta 1 non-catalytic subunit | | |
| 166x | protein kinase, cAMP-dependent, | PRKAR1A | 2.74 |
| | regulatory, type I, alpha | | |
| 167x | protein kinase C, delta | PRKCD | 3.81 |
| 168 | protein kinase C, theta | PRKCQ | 1.23 |
| 169x | phosphatase and tensin homolog | PTEN | 2.01 |
| 170x | PTK6 protein tyrosine kinase 6 | PTK6 | −7.21 |
| 171 | RAB11A, member RAS oncogene | RAB11A | −0.55 |
| | family | | |
| 172x | RAB1A, member RAS oncogene | RAB1A | 2.31 |
| | family | | |
| 173 | RAB24, member RAS oncogene | RAB24 | −0.20 |
| | family | | |
| 174x | RAB33B, member RAS oncogene | RAB33B | −1.83 |
| | family | | |
| 175x | RAB5A, member RAS oncogene | RAB5A | 1.60 |
| | family | | |
| 176x | RAB7A, member RAS oncogene | RAB7A | 2.06 |
| | family | | |
| 177 | ras-related C3 botulinum toxin | RAC1 | 1.37 |
| | substrate 1 (rho family, small | | |
| | GTP binding protein Rac1) | | |
| 178 | v-raf-1 murine leukemia viral | RAF1 | −0.41 |
| | oncogene homolog 1 | | |
| 179 | retinoblastoma 1 | RB1 | 1.32 |
| 180 | RB1-inducible coiled-coil 1 | RB1CC1 | −0.37 |
| 181 | v-rel reticuloendotheliosis viral | RELA | 0.60 |
| | oncogene homolog A (avian) | | |
| 182 | regulator of G-protein signaling | RGS19 | 1.45 |
| | 19 | | |
| 183x | Ras homolog enriched in brain | RHEB | 2.28 |
| 184 | ribosomal protein S6 kinase, | RPS6KB1 | 0.29 |
| | 70 kDa, polypeptide 1 | | |
| 185x | regulatory associated protein of | RPTOR | −1.66 |
| | MTOR, complex 1 | | |
| 186 | SAR1 homolog A (S. cerevisiae) | SAR1A | 1.35 |
| 187 | serpin peptidase inhibitor, | SERPINA1 | −0.86 |
| | clade A (alpha-1 antiproteinase, | | |
| | antitrypsin), member 1 | | |
| 188 | sestrin 2 | SESN2 | 1.17 |
| 189 | SH3-domain GRB2-like endophilin | SH3GLB1 | 0.47 |
| | B1 | | |
| 190x | sirtuin (silent mating type | SIRT1 | −1.63 |
| | information regulation 2 homolog) | | |
| | 1 (S. cerevisiae) | | |
| 191 | sirtuin (silent mating type | SIRT2 | 0.52 |
| | information regulation 2 homolog) | | |
| | 2 (S. cerevisiae) | | |
| 192x | sphingosine kinase 1 | SPHK1 | 1.51 |
| 193 | spinster homolog 1 (Drosophila) | SPNS1 | −1.37 |
| 194x | sequestosome 1 | SQSTM1 | 6.26 |
| 195 | suppression of tumorigenicity 13 | ST13 | 0.83 |
| | (colon carcinoma) | | |
| 196 | serine/threonine kinase 11 | STK11 | −0.04 |
| 197 | TANK-binding kinase 1 | TBK1 | 1.02 |
| 198 | transmembrane 9 superfamily | TM9SF1 | 1.22 |
| | member 1 | | |
| 199x | transmembrane protein 49 | TMEM49 | 2.89 |
| 200x | transmembrane protein 74 | TMEM74 | 2.10 |
| 201 | tumor necrosis factor (ligand) | TNFSF10 | −1.40 |
| | superfamily, member 10 | | |
| 202 | tumor protein p53 | TP53 | −0.17 |
| 203 | tumor protein p53 inducible | TP53INP2 | −0.09 |
| | nuclear protein 2 | | |
| 204x | tumor protein p63 | TP63 | −1.64 |
| 205x | tumor protein p73 | TP73 | −1.66 |
| 206 | tuberous sclerosis 1 | TSC1 | −1.30 |
| 207x | tuberous sclerosis 2 | TSC2 | −1.80 |
| 208 | tumor suppressor candidate 1 | TUSC1 | −1.34 |
| 209 | unc-51-like kinase 1 (C. elegans) | ULK1 | −1.14 |
| 210x | unc-51-like kinase 2 (C. elegans) | ULK2 | 1.51 |
| 211 | unc-51-like kinase 3 (C. elegans) | ULK3 | −1.41 |
| 212 | ubiquitin specific peptidase 10 | USP10 | 1.01 |
| 213 | UV radiation resistance associated | UVRAG | −0.32 |
| | gene | | |
| 214 | vesicle-associated membrane | VAMP3 | 1.04 |
| | protein 3 (cellubrevin) | | |
| 215 | vesicle-associated membrane | VAMP7 | 0.51 |
| | protein 7 | | |
| 216 | vascular endothelial growth | VEGFA | 0.35 |
| | factor A | | |
| 217 | WD repeat and FYVE domain | WDFY3 | −0.05 |
| | containing 3 | | |
| 218 | WD repeat domain 45 | WDR45 | 1.08 |
| 219 | WDR45-like | WDR45L | −1.24 |
| 220x | WD repeat domain, phosphoino- | WIPI1 | 5.28 |
| | sitide interacting 1 | | |
| 221x | WD repeat domain, phosphoino- | WIPI2 | 2.01 |
| | sitide interacting 2 | | |
| 222 | zinc finger, FYVE domain | ZFYVE1 | 1.00 |
| | containing 1 | | |

From the analysis of the present example it has been concluded that about 30% of the analyzed ARGs (70 out of 222) are differentially expressed in the melanoma samples significantly with respect to normal skin samples. It is interesting to note that 10 genes (BAX, CTSB, FOS, HSPA8, ITGA3, ITGA6, MLST8, PTK6, SQSTM1 and WIPI1) show 4-fold greater up- or down-regulation in melanoma samples with respect to the normal skin samples (i.e. fold change difference greater than +4 or lower than −4).

### Example 2

### Comparative Analysis of the Expression of ARGs in Melanoma Vs Nevi Samples

The expression levels of the 222 ARGs evaluated in 45 samples of melanoma and 18 samples of nevi have been taken from the dataset GDS1375 in the GEO database (https://www.ncbi.nlm.nih.gov/gds). Once such expression levels have been obtained, the comparison between the expression levels of the ARGs in melanoma samples and nevi samples has been carried out by means of mean calculation, t test and ROC analysis. The ROC analysis is the most recognized method in binary tests and calculates the area under the curve (AUC) which denotes how effective is the expression of a given gene to discriminate the healthy biopsies (in this case, nevi biopsies) from the melanoma biopsies.

In Table 2 the 42 genes out of the analyzed 222 are reported which resulted to be differentially expressed in a significant relevant way, i.e. with an AUC greater than or equal to 0.85 and p<0.0001.

TABLE 2

| | | Mean | Mean | | | Correlation |
|---|---|---|---|---|---|---|
| | Symbol of | expression in | expression in | | P value | between the gene |
| | the gene | the melanoma | the nevus | AUC | of AUC | and melanoma |
| 1 | ATF4 | 5611 | 7598 | 0.87 | <0.0001 | Yes * |
| 2 | ATG4B | 1016 | 739 | 0.88 | <0.0001 | Yes ** |
| 3x | ATG9A | 1014 | 735 | 0.89 | <0.0001 | No |
| 4x | BAG1 | 727 | 1891 | 1 | <0.0001 | No |
| 5 | BAG3 | 1098 | 1831 | 0.87 | <0.0001 | Yes ** |
| 6 | BAX | 498 | 186 | 0.93 | <0.0001 | Yes * |
| 7 | BCL2 | 1409 | 180 | 0.99 | <0.0001 | Yes ** |
| 8 | BCL2L1 | 1244 | 375 | 0.99 | <0.0001 | Yes ** |
| 9 | BIRC5 | 590 | 230 | 0.92 | <0.0001 | Yes * |
| 10x | CAPN2 | 9236 | 5096 | 0.96 | <0.0001 | No |
| 11 | CAPNS1 | 8826 | 4392 | 0.93 | <0.0001 | Yes ** |
| 12 | CDKN1A | 2790 | 1397 | 0.90 | <0.0001 | Yes ** |
| 13 | CDKN2A | 650 | 335 | 0.86 | <0.0001 | Yes * |
| 14 | CFLAR | 472 | 796 | 0.85 | <0.0001 | Yes ** |
| 15x | CHMP2B | 282 | 561 | 0.87 | <0.0001 | No |
| 16 | CTSB | 16713 | 1655 | 0.99 | <0.0001 | Yes * |
| 17 | CTSD | 2115 | 1029 | 0.89 | <0.0001 | Yes * |
| 18 | CX3CL1 | 266 | 627 | 0.91 | <0.0001 | Yes * |
| 19 | EGFR | 184 | 1976 | 0.98 | <0.0001 | Yes ** |
| 20 | EIF2AK3 | 566 | 282 | 0.93 | <0.0001 | Yes * |
| 21 | EIF2S1 | 16903 | 9247 | 0.90 | <0.0001 | Yes * |
| 22 | ERBB2 | 2107 | 1695 | 0.90 | <0.0001 | Yes ** |
| 23 | FAS | 338 | 681 | 0.89 | <0.0001 | Yes * |
| 24 | FOXO1 | 482 | 1055 | 0.96 | <0.0001 | Yes ** |
| 25x | GNAI3 | 193 | 362 | 0.94 | <0.0001 | No |
| 26 | HDAC1 | 1614 | 1146 | 0.86 | <0.0001 | Yes * |
| 27 | HSPA5 | 3830 | 2390 | 0.86 | <0.0001 | Yes * |
| 28 | HSPB8 | 200 | 947 | 0.94 | <0.0001 | Yes ** |
| 29 | ITGA3 | 2436 | 497 | 0.95 | <0.0001 | Yes * |
| 30x | ITGB4 | 206 | 944 | 0.87 | <0.0001 | No |
| 31x | KIAA0226 | 186 | 104 | 0.85 | <0.0001 | No |
| 32 | MAPK1 | 730 | 1339 | 0.86 | <0.0001 | Yes * |
| 33x | MLST8 | 833 | 453 | 0.90 | <0.0001 | No |
| 34 | NFE2L2 | 1410 | 2622 | 0.91 | <0.0001 | Yes * |
| 35 | PARP1 | 2212 | 975 | 0.99 | <0.0001 | Yes * |
| 36 | PEA15 | 5307 | 3477 | 0.94 | <0.0001 | Yes ** |
| 37x | PEX3 | 343 | 670 | 0.93 | <0.0001 | No |
| 38 | PTK6 | 63 | 556 | 0.96 | <0.0001 | Yes ** |
| 39 | SQSTM1 | 4197 | 2636 | 0.95 | <0.0001 | Yes * |
| 40 | TP63 | 131 | 1067 | 0.93 | <0.0001 | Yes * |
| 41 | TP73 | 578 | 785 | 0.89 | <0.0001 | Yes * |
| 42x | WIPI1 | 3043 | 374 | 0.99 | <0.0001 | No |

Through the analysis by means of the Chilibot software on Pubmed, it turned out that 10 genes out of the 42 reported in Table 2 have never been correlated to the diagnosis or prognosis of melanomas. Such genes are: ATG9A, BAG1, CAPN2, CHMP2B, GNAI3, ITGB4, KIAA0226, MLST8, PEX3, WIPI1 (indicated by an x in the Table 2 shown above).

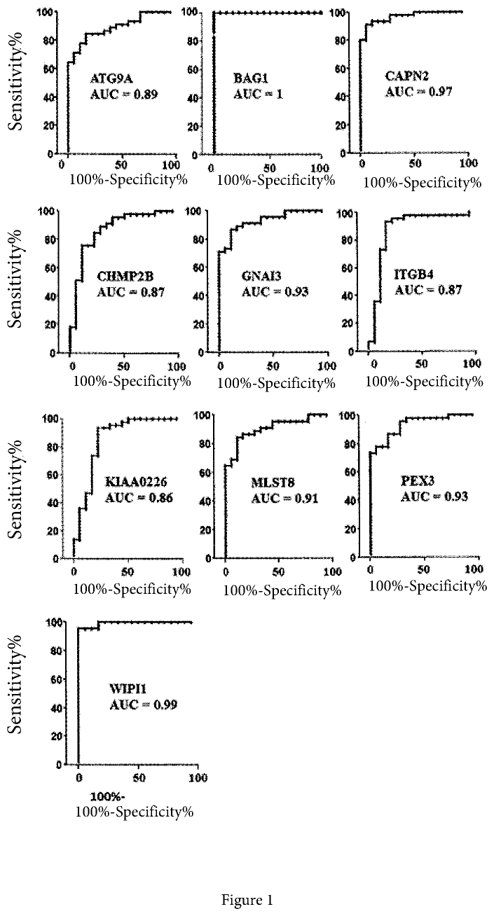

The ROC curves of each of these 10 genes are depicted in FIG. 1; in particular, in each plot in FIG. 1, it is possible to note on the x axis the specificity %, and on the y axis the sensitivity %.

### Example 3

### Validation of the Data of Gene Expression on the IST Online Database

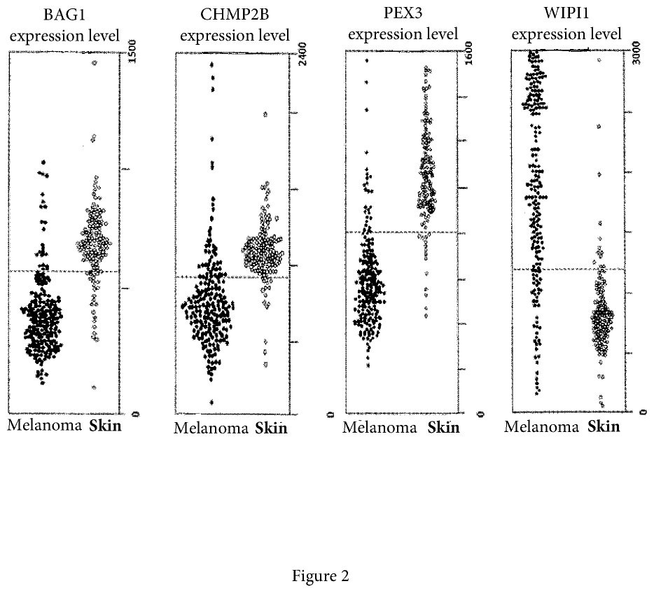

The expression of the 10 genes indicated by an x in Table 2 has been validated by means of a set of expression data of 208 melanoma samples and 147 samples of healthy skin taken from the IST Online database (http://ist.medisapiens.com/), which expresses the expression data as scatter plots. From the analysis of the dataset of IST Online, it has been noted that 4 genes out of the validated 10 have high expression differences in the melanoma with respect to the healthy skin: BAG1, CHMP2B, PEX3 and WIPI1. The scatter plots obtained for BAG1, CHMP2B, PEX3 and WIPI1 have been de novo graphically replotted and are depicted in FIG. 2. In particular, in FIG. 2 it is possible to observe, for each of the 4 genes, the gene expression data detected on melanoma biopsies on the left, and the corresponding gene expression data detected on biopsies of healthy skin on the right. From the analysis of the present example, the gene expression of BAG1, CHMP2B and PEX3 in the melanoma is lower than that of the healthy skin in at least 90% of the melanoma cases, while the gene expression of WIPI1 in the melanoma is greater than that in the healthy skin in at least 90% of the melanoma cases.

### Example 4

### Evaluation of the Expression Levels of the BAG1, CHMP2B, PEX3 and WIPI1 Proteins on Histological Sections

The expression level of the proteins of the 4 validated genes BAG1, CHMP2B, PEX3 and WIPI1 has been analyzed on the basis of the histochemical data reported in the Human Protein Atlas (HPA) website (https://www.proteinatlas.org), which contains images of histological sections wherein the proteins are highlighted by means of a bond with labeled antibodies. The expression of the proteins deriving from the 4 identified genes in the previous example has been evaluated thanks to the analysis of 80 images from histological sections (in particular, from 47 images of melanoma and 33 images of healthy skin). The 80 images of the histological sections of HPA have been converted in grey scale by means of the photo editing GNU Image Manipulation Program (GIMP) software, which allowed to obtain quantification of the pixel distribution.

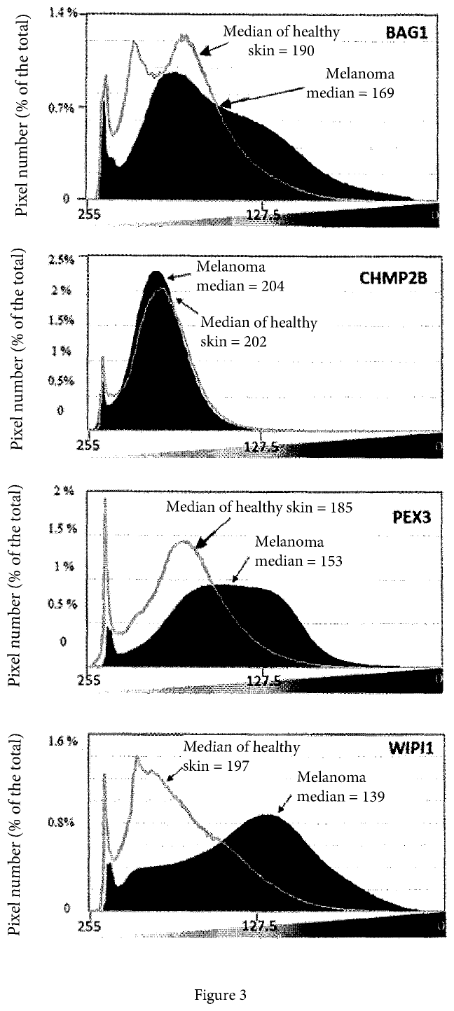

In FIG. 3 the plots relative to the expression level of each of the 4 proteins analyzed in melanoma and healthy skin samples are depicted. In each of such plots, the dark curves refer to the melanoma samples and the light curves refer to the control samples. In three cases the medians of the dark curves are shifted to the right with respect to the medians of the light curves, thus suggesting a greater expression in the melanoma, for the BAG1, PEX3 and WIPI1 proteins. Such plots have been obtained starting from the quantification of the pixel distribution, and in particular have been obtained by carrying out the following steps:
1) selection of the image;
2) removal of the background (by means of the conversion of the background from white into transparent);
3) conversion of the RGB image color into greyscale; and
4) production of histograms.

The information referring to the mean, pixels, median and standard deviation present in FIG. 3 has been obtained by selecting, in the image menu of GIMP: Color→Information→Histogram.

The expression of the CHMP2B protein results to be unchanged in the melanoma with respect to the normal tissues.

It should be noted that the gene expression levels of BAG1 and PEX3 (detected in the validation of Example 3) and the total level of the proteins encoded by them (evaluated in the present Example) do not correspond, i.e. the gene expression of BAG1 and PEX3 is lower in the melanoma with respect to the healthy skin, however the expression of the proteins encoded by them is greater in the melanoma with respect to the healthy skin. Such contradiction is only apparent, indeed the gene expression levels depend on the transcription mechanism, while the protein expression levels are based on the reactive adaptation of the synthesis/degradation mechanism of the proteins in response to the modified gene expression levels. On the other hand the expression levels of WIPI1 are increased in the melanoma samples with respect to the healthy skin samples, both at the level of gene transcription (see Example 3) and of protein expression (Example 4).

### Example 5

### Evaluation of the Prognostic Value of the Considered Genes

The prognostic value of the BAG1, CHMP22B, PEX3 and WIPI1 genes has been evaluated by means of the data reported in HPA relative to patients with melanoma with 3 years-survival data. Such patients have been stratified based on the high or low expression level of the 4 genes under evaluation, and then the progression, positive or negative, of the tumoral pathology (based on the survival of the patient) has been associated to each so-stratified patient. This way, it has been possible to associate the high or low expression level of each of the 4 genes with a higher or lower survival of the patients.

From the evaluation of the present example, the genes with prognostic value, among the four considered genes, are BAG1 and WIPI1. In particular: the analysis of the survival in 102 patients with melanoma showed a prognostic value indicating a greater survival in the patients in which the BAG1 expression level in the melanoma is low (p=0.04). The survival analysis in 102 patients with melanoma showed that WIPI1 is a favorable prognostic marker in melanoma (p<0.0001).

1. A method to perform a diagnosis of melanoma in a subject comprising the following steps:
a) determining the expression levels of at least one gene, and/or of at least one protein encoded by at least one gene, from one or more samples taken and isolated from said subject; and
b) comparing said expression levels determined in step a), with the standard or control expression levels of the same genes and/or the same proteins
characterized in that said at least one gene is selected from the group of genes: ATG9A, BAG1, CAPN2, CHMP2B, GNAI3, ITGB4, KIAA0226, MLST8, PEX3, and WIPI1, and/or said at least one protein is a protein encoded by the genes selected from the group of genes: BAG1, PEX3, and WIPI1.

2. The method according to claim 1, characterized in that said at least one gene of which said expression levels are determined is selected from the group of genes: BAG1, CHMP2B, PEX3, and WIPI1.

3. The method to perform a prognosis for a subject suffering from melanoma comprising the following steps:
a′) determining the expression levels of at least one gene, and/or of at least one protein encoded by a gene, from at least one sample taken and isolated from said subject; and
b′) comparing said expression levels determined in step a′), with the standard or control expression levels of the same genes and/or the same proteins
characterized in that said at least one gene is selected from the BAG1 and WIPI1 genes, and/or said at least one protein is a protein encoded by the genes selected from BAG1 and WIPI1.

4. The method according to any one of the preceding claims, characterized in that said at least one gene of which said expression levels are determined is WIPI1, and/or said protein of which said expression levels are determined is the protein encoded by the WIPI1 gene.

5. The method according to any one of the preceding claims, characterized in that said at least one isolated sample is a sample of isolated epidermis.

6. The method according to any one of the preceding claims, characterized in that said at least one isolated sample is a sample of isolated nevus.

7. The method according to claims 1 to 4, characterized in that said at least one isolated sample is a sample of isolated blood.

8. A kit comprising one or more agents suitable to measure the expression levels of at least one gene and/or at least one protein, characterized in that said at least one gene is selected from the group of genes: ATG9A, BAG1, CAPN2, CHMP2B, GNAI3, ITGB4, KIAA0226, MLST8, PEX3, and WIPI1, and/or said at least one protein is a protein encoded by the genes selected from the group of genes: BAG1, PEX3, and WIPI1.

9. The kit according to the preceding claim, characterized in that said at least one gene is selected from the group of genes: BAG1, CHMP2B, PEX3, and WIPI1.

10. The kit according to claim 9, characterized in that said at least one gene is selected from the BAG1 and WIPI1 genes, and/or said at least one protein is a protein encoded by the genes selected from BAG1 and WIPI1.
